(19) United States
(12) Patent Application Publication
(10) Pub. No.: US 2019/0365843 A1
(43) Pub. Date: Dec. 5, 2019

(54) Antimicrobial Activity of Bioactive Compounds and Their Derivatives

(71) Applicants: Anju Majeed, East Windsor, NJ (US); Muhammed Majeed, Edison, NJ (US); Kalyanam Nagabhushanam, East Windsor, NJ (US); Nooruddin Thajuddin, Tiruchirapalli (IN); Sivakumar Arumugam, Bangalore (IN); Krishnamurthy Kulithalai Viswanathan, Bangalore (IN); Samuel Thomas Manoharan, Bangalore (IN); Furqan Ali, Bangalore (IN); John Adams Sebastian, Bangalore (IN)

(72) Inventors: Anju Majeed, East Windsor, NJ (US); Muhammed Majeed, Edison, NJ (US); Kalyanam Nagabhushanam, East Windsor, NJ (US); Nooruddin Thajuddin, Tiruchirapalli (IN); Sivakumar Arumugam, Bangalore (IN); Krishnamurthy Kulithalai Viswanathan, Bangalore (IN); Samuel Thomas Manoharan, Bangalore (IN); Furqan Ali, Bangalore (IN); John Adams Sebastian, Bangalore (IN)

(21) Appl. No.: 15/990,972

(22) Filed: May 29, 2018

(51) Int. Cl. A61K 36/9066; A61K 31/12; A61K 31/222; A61K 31/216; A61K 31/265; A61K 31/42; A61K 31/133; A61P 31/04

(52) U.S. Cl. CPC A61K 36/9066; A61K 31/12; A61K 31/222; A61P 31/04; A61K 31/265; A61K 31/42; A61K 31/133; A61K 31/216

(57) ABSTRACT

Disclosed are the antimicrobial effects of bioactive compounds isolated from Curcuma species and their derivatives. Specifically, the invention discloses the growth inhibition of and management of infections caused by Enteroaggregative Escherichia coli (EAEC), Enterotoxigenic Escherichia coli (ETEC) and Pleisiomonas species by curcuminoids, calebin A and their derivatives.

### CROSS-REFERENCE TO RELATED PATENT APPLICATION

The present invention is non-provisional filing of U.S. provisional patent application No. 62/512,271 filed on 30 May 2017.

### BACKGROUND OF THE INVENTION

### Field of the Invention

The invention in general relates to antimicrobial activity of bioactive compounds and their derivatives. More specifically the present invention relates to antimicrobial activity of curcuminoids, calebin A and their derivatives.

### Description of Prior Art

Recently, alternative medicine has shown great growth potential worldwide. Bioactive molecules, isolated from plant based sources are now being widely used as a dietary supplement for maintaining general health and in the management of many diseases and disorders. Curcuma longa, commonly known as turmeric has long been used in Ayurvedic, Siddha and Unani systems for its wide range of biological activity. Curcumin and other active of turmeric viz turmerin, turmerone, elemene, furanodiene, curdione, bisacurone, cyclocurcumin, calebin A, and germacrone are reported to have wide range of therapeutic activities. Turmeric is known for its antioxidant, anti-inflammatory, anticancer, antigrowth, anti-arthritic, anti-atherosclerotic, antidepressant, anti-aging, antidiabetic, antimicrobial, wound healing, and memory-enhancing activities for many years. Some of the benefits of turmeric are outlined in the following prior art documents
1. Niazi et al., (2010) Pharmacotherapeutics of Curcuma longa—A Potent Patent, IJPPR, 1(1):24-33.
2. Aggarwal et al., (2013). Curcumin-free turmeric exhibits anti-inflammatory and anticancer activities: Identification of novel components of turmeric, Mol Nutr Food Res. 57(9): 1529-1542.
3. Top 10 turmeric benefits, OMNIBIOTICS, Jan. 21, 2018, http://omnibiotics.com/turmeric-benefits-top-10-list/(Accessed on 21 May 2018)

Although the antimicrobial activity of curcuminoids has been reported in the following prior arts:
1. Teow et al., (2016) Antibacterial Action of Curcumin against Staphylococcus aureus: A Brief Review, Journal of Tropical Medicine, 2016:1-10 http://dx.doi.org/10.1155/2016/2853045
2. Luo et al., (2014) demethoxycurcumin: a potential antimicrobial agent, Therm Anal Calorim, 115:2331. https://doi.org/10.1007/s 10973-013-3103-6.
3. Singh et al., (2012) Evaluation of antimicrobial activity of curcuminoids isolated from turmeric, Int. J. of Pharm. & Life Sci., 3(1):1368-1376.
4. Moghadamtousi et al., (2014) A Review on Antibacterial, Antiviral, and Antifungal Activity of Curcumin, Biomed Res Int. 2014:186864.
   the anti-microbial spectrum of curcuminoids differ for the different species and strains of microbes. Moreover, the anti-microbial activity of calebin A has not been reported yet. The present invention solves the above mentioned problem by disclosing the anti-microbial activity of bioactive compounds isolated from Curcuma species and their derivatives.

The principle objective of the invention is to disclose the anti-microbial effect of bioactive compounds isolated from Curcuma species and their derivatives against wide range of microbes by inhibiting the growth of said microbes.

It is another objective of the invention to disclose a method of therapeutic management of infections caused by microbes using bioactive compounds isolated from Curcuma species and their derivatives.

### SUMMARY OF THE INVENTION

The present invention discloses the antimicrobial effects of bioactive compounds isolated from Curcuma species and their derivatives. Specifically, the invention discloses the growth inhibition of and management of infections caused by Enteroaggregative E. coli (EAEC), Enterotoxigenic E. coli (ETEC) and Pleisiomonas species by curcuminoids, calebin A and their derivatives.

### BRIEF DESCRIPTION OF THE FIGURES

FIG. 1 is a representative image of the culture plate showing the zone of growth inhibition of Staphylococcus aureus by curcumin and demethoxycurcumin

FIG. 2 is a representative image of the culture plate showing the zone of growth inhibition of Proteus species by curcumin, demethoxycurcumin and bisdemethoxycurcumin.

FIG. 3 is a representative image of the culture plate showing the zone of growth inhibition of EAEC by calebin A, O,O′-diacetylcalebin-A, diethylcalebin-A dicarbonate, and tetrahydrocurcumin Isoxazole.

FIG. 4a is a representative image of the culture plate showing the zone of growth inhibition of ETEC by curcumin, demethoxycurcumin, bis-demethoxycurcumin, O,O′-diacetylcurcumin, and calebin A.

FIG. 4b is a representative image of the culture plate showing the zone of growth inhibition of ETEC by O,O′-diacetylcalebin-A, diethylcalebin-A dicarbonate, tetrahydrocurcumin Isoxazole, Aminotetrahydrocurcumin and dibromocurcumin.

FIG. 5 is a representative image of the culture plate showing the zone of growth inhibition of Vibrio cholerae by curcumin.

FIG. 6 is a representative image of the culture plate showing the zone of growth inhibition of Aeromonas species by demethoxycurcumin.

FIG. 7a is a representative image of the culture plate showing the zone of growth inhibition of Pleisiomonas species by Curcumin, demethoxycurcumin and bis-demethoxycurcumin.

FIG. 7b is a representative image of the culture plate showing the zone of growth inhibition of Pleisiomonas species by Demethoxycalebin-A, and bisdemethoxycalebin-A.

### DESCRIPTION OF THE MOST PREFERRED EMBODIMENTS

In the most preferred embodiment, the present invention discloses a method of inhibiting the growth of E. coli strains, said method comprising steps of bringing into contact E. coli strains with effective concentration of bioactive compounds isolated from Curcuma species and their derivatives, individually or in combination to bring about inhibition in the growth of E. coli In a related embodiment, the strains of E. coli include Enteroaggregative E. coli (EAEC) and Enterotoxigenic E. coli (ETEC). In a related embodiment, the bioactive compounds and their derivatives for the inhibiting the growth of EAEC are selected from the group consisting of calebin A, O,O′-diacetylcalebin-A, diethylcalebin-A dicarbonate, and Tetrahydrocurcumin Isoxazole. In a related embodiment, the bioactive compounds and their derivatives for the inhibiting the growth of ETEC are selected from the group consisting of Curcumin, demethoxycurcumin, bis-demethoxycurcumin, O,O′-diacetylcurcumin, calebin A, O,O′-diacetylcalebin-A, diethylcalebin-A dicarbonate, TetrahydrocurcuminIsoxazole, Amino-Tetrahydrocurcumin and dibromocurcumin.

In another preferred embodiment, the present invention discloses a method for the therapeutic management of infections caused by strains of E. coli. in mammals, said method comprising step of administering an effective dose of bioactive compounds isolated from Curcuma species and their derivatives, individually or in combination, to said mammals to bring about reduction in the infection caused by E. coli strains. In a related embodiment, the strains of E. coli include Enteroaggregative E. coli (EAEC) and Enterotoxigenic E. coli (ETEC). In a related embodiment, the bioactive compounds and their derivatives for treating EAEC infections are selected from the group consisting of calebin A, O,O′-diacetylcalebin-A, diethylcalebin-A dicarbonate, and tetrahydrocurcumin Isoxazole. In a related embodiment, the bioactive compounds and their derivatives for treating ETEC infections are selected from the group consisting of Curcumin, demethoxycurcumin, bis-demethoxycurcumin, O,O′-diacetylcurcumin, calebin A, O,O′-diacetylcalebin-A, diethylcalebin-A dicarbonate, tetrahydrocurcumin Isoxazole, Amino tetrahydrocurcumin and dibromocurcumin. In another related embodiment, the infections caused by EAEC are selected from the group consisting of watery diarrhea, mucoid diarrhea, low-grade fever, nausea, tenesmus, and borborygmi. In another related embodiment, the infections caused by ETEC are selected from the group consisting of severe diarrhea, dysentery, abdominal cramps, and fever. In yet another related embodiment, the mammal is human

In the most preferred embodiment, the present invention discloses a method of inhibiting the growth of Pleisiomonas species, said method comprising steps of bringing into contact Pleisiomonas species with effective concentration of bioactive compounds isolated from Curcuma species and their derivatives, individually or in combination to bring about inhibition in the growth of Pleisiomonas species In a related embodiment, the bioactive compounds and their derivatives are selected from the group consisting of Curcumin, demethoxycurcumin, bis-demethoxycurcumin, diethylcalebin-A dicarbonate, Demethoxycalebin-A, and bisdemethoxycalebin-A.

In another preferred embodiment, the present invention a method for the therapeutic management of infections caused by strains of Pleisiomonas species in mammals, said method comprising step of administering an effective dose of bioactive compounds isolated from Curcuma species and their derivatives, individually or in combination, to said mammals to bring about reduction in the infection caused by Pleisiomonas species In a related embodiment, the bioactive compounds and their derivatives are selected from the group consisting of Curcumin, demethoxycurcumin, bis-demethoxycurcumin, diethylcalebin-A dicarbonate, Demethoxycalebin-A, and bisdemethoxycalebin-A. In another related embodiment, the infections caused by Pleisiomonas species are selected from the group consisting of gastrointestinal infections, extraintestinal infections, gastroenteritis, watery diarrhea, abdominal pain, nausea and/or vomiting, headache, dehydration and fever. In yet another related embodiment, the mammal is human.

The specific examples included herein below illustrate the aforesaid most preferred embodiments of the present invention.

### Example 1: Methodology

The present invention was aimed at evaluating the antimicrobial activity spectrum of bioactive compounds and their derivatives as mentioned herein below (Table 1).

TABLE 1
Bioactive compounds and derivatives

| SI. | | Molecular | |
|---|---|---|---|
| No | Sample name | weight | Structure |
| 1 | Curcumin | 368.39 | 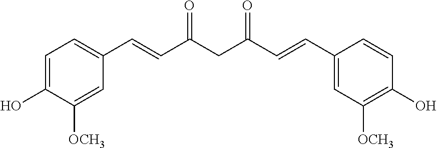 [SMILES: COc1cc(/C=C/C(=O)CC(=O)/C=C/c2ccc(O)c(OC)c2)ccc1O] |
| 2 | Demethoxycurcumin (DMC) | 338.36 | 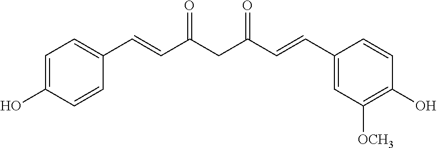 [SMILES: COc1cc(/C=C/C(=O)CC(=O)/C=C/c2ccc(O)cc2)ccc1O] |
| 3 | Bis- demethoxycurcumin (BDMC) | 308.34 | 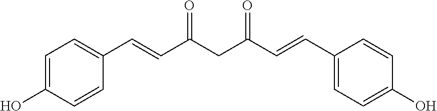 [SMILES: O=C(/C=C/c1ccc(O)cc1)CC(=O)/C=C/c1ccc(O)cc1] |
| 4 | O,O′- Diacetylcurcumin | 452.47 | 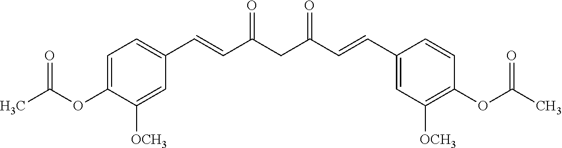 [SMILES: COc1cc(/C=C/C(=O)CC(=O)/C=C/c2ccc(OC(C)=O)c(OC)c2)ccc1OC(C)=O] |
| 5 | Calebin-A | 384.39 | 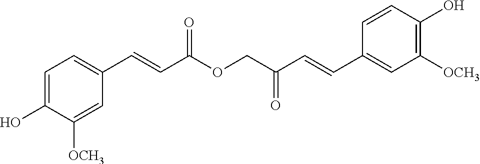 [SMILES: COc1cc(/C=C/C(=O)COC(=O)/C=C/c2ccc(O)c(OC)c2)ccc1O] |
| 6 | O,O′- Diacetylcalebin-A | 468.47 | 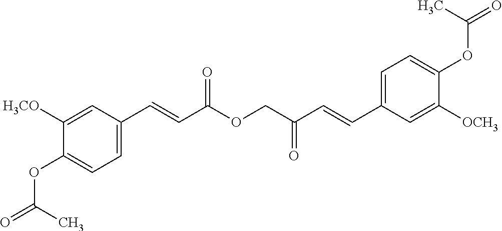 [SMILES: COc1cc(/C=C/C(=O)COC(=O)/C=C/c2ccc(OC(C)=O)c(OC)c2)ccc1OC(C)=O] |
| 7 | Diethylcalebin-A dicarbonate | 528.52 | 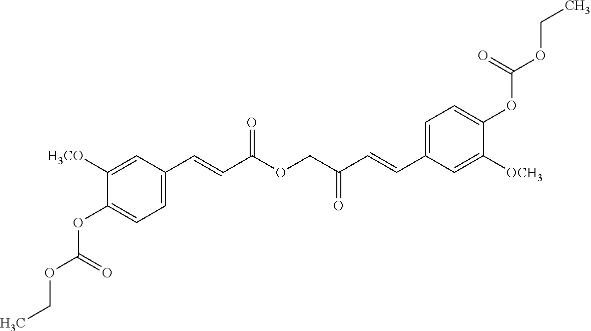 [SMILES: CCOC(=O)Oc1ccc(/C=C/C(=O)COC(=O)/C=C/c2ccc(OC(=O)OCC)c(OC)c2)cc1OC] |
| 8 | Tetrahydrocurcumin Isoxazole | 369.42 | 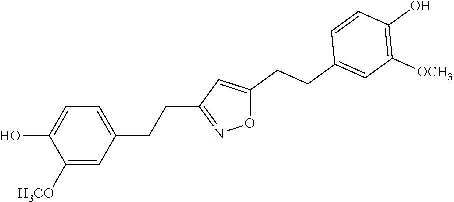 [SMILES: COc1cc(CCc2cc(CCc3ccc(O)c(OC)c3)on2)ccc1O] |
| 9 | Amino- tetrahydrocurcumin | 371.44 | 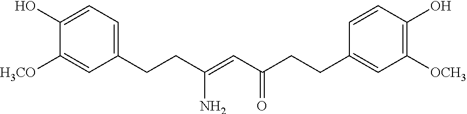 [SMILES: COc1cc(CCC(=O)/C=C(\N)CCc2ccc(O)c(OC)c2)ccc1O] |
| 10 | Dibromocurcumin | 430.19 | 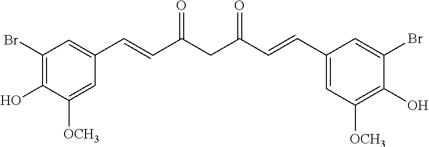 [SMILES: COc1cc(/C=C/C(=O)CC(=O)/C=C/c2cc(Br)c(O)c(OC)c2)cc(Br)c1O] |
| 11 | Demethoxycalebin- A1 | 354.36 | 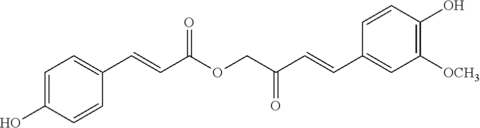 [SMILES: COc1cc(/C=C/C(=O)COC(=O)/C=C/c2ccc(O)cc2)ccc1O] |
| 12 | Demethoxycalebin- A2 | 354.36 | 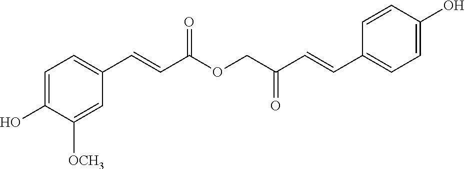 [SMILES: COc1cc(/C=C/C(=O)OCC(=O)/C=C/c2ccc(O)cc2)ccc1O] |
| 13 | Bisdemethoxycalebin- A | 324.34 | 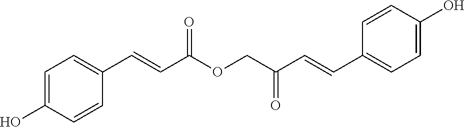 [SMILES: O=C(/C=C/c1ccc(O)cc1)COC(=O)/C=C/c1ccc(O)cc1] |
| 14 | Control blank | — | |

The different bioactive compounds isolated from Curcuma species and their derivatives are obtained from Sami Labs limited, Bangalore, India. Bioactive compounds 1-10 and 11-14 were screened for antimicrobial spectrum of activity at concentration ranges 150 mg-100 mg/ml and 100 mg-50 mg/ml of test diluent respectively.

Strains:

Among the bacterial agents tested Gram positive and Gram negative bacterial groups represented by Staphylococcus aureus and Enterococcus species and pathogenic members of Enterobacteriaceae, Vibronaceae, Aeromonadaceae, Pseudomonadaceae were tested. Freshly sub-cultured bacterial strains were inoculated into peptone water and incubated for 4 hours at 37° C. and adjusted to a turbidity of 0.5 (Gram Negative Bacilli), 1 (Gram Positive Cocci) and 4 (Pus cells, GNB, & few Budding yeast cells) Mc Farland standards (10⁸ CFU/ml) respectively.

Preparation of Bioactive Compounds:

The different bioactive compounds and their derivatives were dissolved and diluted with solvents (DMSO), number of subsequent dilutions was performed to obtain different concentrations of the bioactive compounds and their derivatives. (Working Concentrations).

Agar Well Diffusion Method:

Agar well diffusion method is widely used to evaluate the antimicrobial activity of plants or other synthetic products. The suspension (microbial inoculum) was used to inoculate into Muller Hinton Agar Petri plates by lawn culture. Well (diameter 6 mm) were punched in the agar by sterile borer and filled with 50 μl of dissolved extracts. Plates were incubated in incubator at 37° C. for 24 hours and measured the growth inhibition zone diameters in mm compared with that of control (DMSO) showing no growth. The antimicrobial agent diffuses in the agar medium and inhibits the growth of the microbial strain tested (Valgas et al., (2016) Methods for in vitro evaluating antimicrobial activity: A review. Journal of Pharmaceutical Analysis. 6(2):71-9).

Agar Disk-Diffusion Method:

The bioactive compounds were tested with desired concentrations in 6 mm filter paper discs (Whatman, no. 3) were impregnated with 10 μL of each of the different dilutions. The discs were allowed to remain at room temperature until complete diluent evaporation and kept under refrigeration until ready to be used. 10 μL of diluents used to products were used as control. Tests were performed by Agar disk-diffusion testing method, which is the official method used in many clinical microbiology laboratories for routine antimicrobial susceptibility testing.

Similar to the procedure used in Agar well diffusion method, the suspension (microbial inoculum) was used to inoculate into Muller Hinton Agar Petri plates by lawn culture. Then, filter paper discs (about 6 mm in diameter), containing the test compound at a desired concentration, were placed on the agar surface. The Petri dishes were incubated under suitable incubation conditions. Generally, antimicrobial agent diffuses into the agar and inhibits germination and growth of the test microorganism and then the diameters of inhibition growth zones are measured (Valgas et al., (2007) Screening methods to determine antibacterial activity of natural products. Brazilian Journal of Microbiology. 38:369-80; Balouiri M et al., (2016) Methods for in vitro evaluating antimicrobial activity: A review. Journal of Pharmaceutical Analysis. 6(2):71-9).

Bioactive compounds showing zone of inhibition beyond the size of 6 mm are considered to be having an anti-microbial activity.

### Example 2: Results

The results of the anti-microbial activity of the bioactive compounds are mentioned in Table 2.

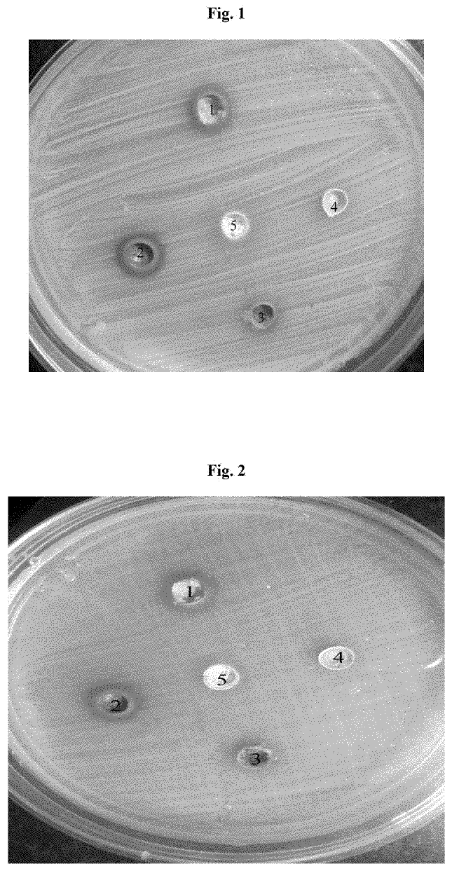

The results indicated that S. aureus was inhibited by curcumin and demethoxycurcumin with a zone of inhibition of 10 mm and 11 mm respectively (FIG. 1). The other bioactive compounds did not show any inhibition in the growth of S aureus. Curcuminoids, containing curcumin, demethoxy curcumin and bisdemethoxycurcumin are already reported to inhibit the growth of S. aureus (Teow et al., (2016) Antibacterial Action of Curcumin against Staphylococcus aureus: A Brief Review, Journal of Tropical Medicine, 2016:1-10 http://dx.doi.org/10.1155/2016/2853045).

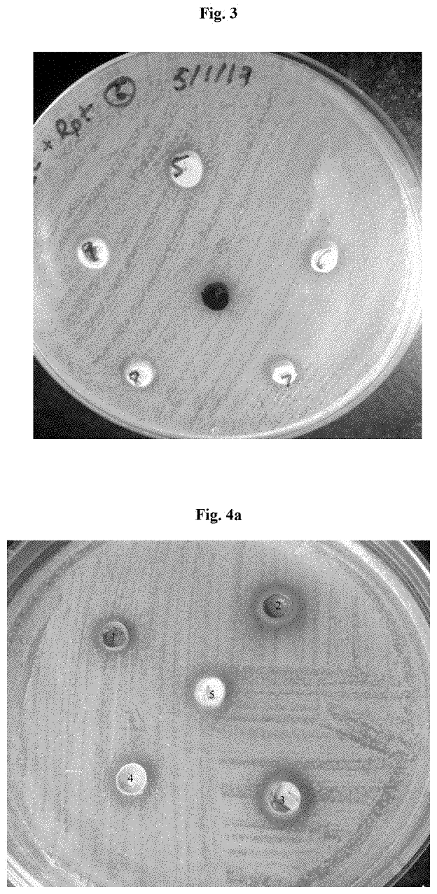

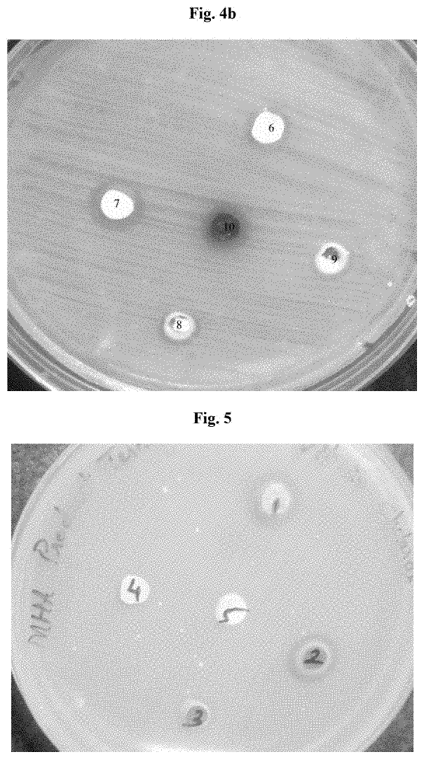

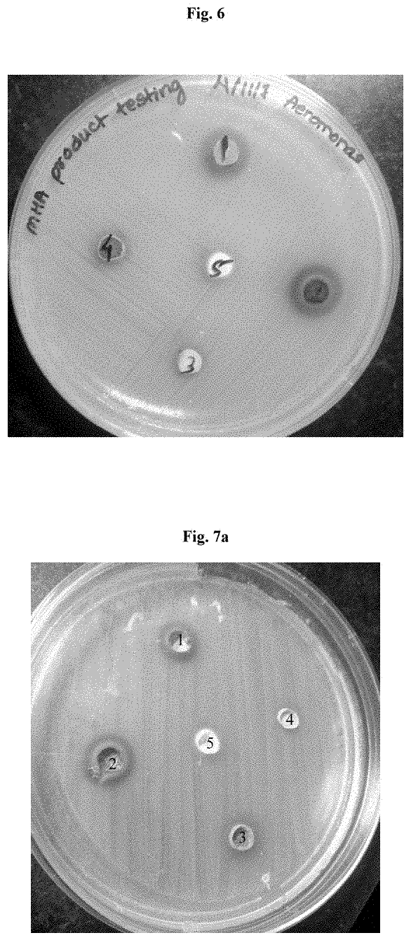

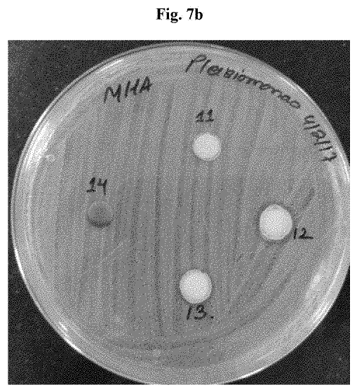

The growth of Proteus species was inhibited by curcumin, demethoxy curcumin and bisdemethoxycurcumin with a zone of inhibition of 11 mm, 12 mm and 7 mm respectively at both concentrations of 100 mg and 150 mg (FIG. 2). The bioactive compounds, Curcumin, demethoxycurcumin, bis-demethoxycurcumin, O,O′-diacetylcurcumin, calebin A, O,O′-diacetylcalebin-A, diethylcalebin-A dicarbonate, tetrahydrocurcumin Isoxazole, Amino tetrahydrocurcumin and dibromocurcumin significantly inhibited the growth of ETEC (FIG. 3). ETEC is a leading cause of diarrhea in children with the development of diarrhea, dysentery, abdominal cramps, and fever but releasing toxins (Qadri et al., (2005) Enterotoxigenic Escherichia coli in Developing Countries: Epidemiology, Microbiology, Clinical Features, Treatment, and Prevention, Clin Microbiol Rev. 18(3): 465-483). The above mentioned bioactive compounds and their derivative can be used effectively for treating the infections of ETEC. Another growth of another type of E. coli, EAEC, was inhibited by calebin A, O,O′-diacetylcalebin-A, diethylcalebin-A dicarbonate (FIGS. 4a and 4b), indicating that it can be used to treat the infections caused by EAEC (Jensen et. al., (2014) Epidemiology and Clinical Manifestations of Enteroaggregative Escherichia coli, Clinical Microbiology Reviews 27(3):614-630). Curcumin and demethoxycurcumin inhibited the growth of Vibrio cholerae (FIG. 5) and Aeromonas species (FIG. 6) respectively and the growth of Plesiomonas species was inhibited by Curcumin, demethoxycurcumin, bis-demethoxycurcumin, diethylcalebin-A dicarbonate, Demethoxycalebin-A, and bisdemethoxycalebin-A (FIGS. 7a and 7b), indicating that the respective bioactive compounds may be administered to treat the corresponding infections. The bioactive compounds did not significantly inhibit the growth of Enterococcus species, Salmonella typhimurium, Shigella species, Pseudomonas species, Klebsiella species, Candida albicans and Citrobacter species

While the invention has been described with reference to a preferred embodiment, it is to be clearly understood by those skilled in the art that the invention is not limited thereto. Rather, the scope of the invention is to be interpreted only in conjunction with the appended claims.

TABLE 2
Results of the anti-microbial activity of the bioactive compounds and their derivatives.

| | 1 | | 2 | | 3 | | 4 | | 5 | | 6 | | 7 | | 8 | | 9 | | 10 | | 11 | | 12 | | 13 | | 14 | |
|---|---|---|---|---|---|---|---|---|---|---|---|---|---|---|---|---|---|---|---|---|---|---|---|---|---|---|---|---|
| | 150 | 100 | 150 | 100 | 150 | 100 | 150 | 100 | 150 | 100 | 150 | 100 | 150 | 100 | 150 | 100 | 150 | 100 | 150 | 100 | 150 | 100 | 150 | 100 | 150 | 100 | 150 | 100 |
| | mg | mg | mg | mg | mg | mg | mg | mg | mg | mg | mg | mg | mg | mg | mg | mg | mg | mg | mg | mg | mg | mg | nag | mg | mg | mg | mg | mg |
| S. aureus | 10 | 10 | 11 | 11 | 6 | 6 | 6 | 6 | 6 | 6 | 6 | 6 | 6 | 6 | 6 | 6 | 6 | 6 | 6 | 6 | NA | 6 | NA | 6 | NA | 6 | NA | 6 |
| Enterococcus | 6 | 6 | 6 | 6 | 6 | 6 | 6 | 6 | 6 | 6 | 6 | 6 | 6 | 6 | 6 | 6 | 6 | 6 | 6 | 6 | NA | 6 | NA | 6 | NA | 6 | NA | 6 |
| sp. | | | | | | | | | | | | | | | | | | | | | | | | | | | | |
| Salmonella | 6 | 6 | 6 | 6 | 6 | 6 | 6 | 6 | 6 | 6 | 6 | 6 | 6 | 6 | 6 | 6 | 6 | 6 | 6 | 6 | NA | 6 | NA | 6 | NA | 6 | NA | 6 |
| typhimurium | | | | | | | | | | | | | | | | | | | | | | | | | | | | |
| Proteus sp. | 11 | 11 | 12 | 12 | 7 | 7 | 6 | 6 | 6 | 6 | 6 | 6 | 6 | 6 | 6 | 6 | 6 | 6 | 6 | 6 | NA | 6 | NA | 6 | NA | 6 | NA | 6 |
| E. coli ATCC | 6 | 6 | 6 | 6 | 6 | 6 | 6 | 6 | 6 | 6 | 6 | 6 | 6 | 6 | 6 | 6 | 6 | 6 | 6 | 6 | NA | 6 | NA | 6 | NA | 6 | NA | 6 |
| 25922 | | | | | | | | | | | | | | | | | | | | | | | | | | | | |
| EAEC | 6 | 6 | 6 | 6 | 6 | 6 | 6 | 6 | 7 | 7 | 7 | 7 | 9 | 9 | 8 | 8 | 6 | 6 | 6 | 6 | NA | 6 | NA | 6 | NA | 6 | NA | 6 |
| ETEC | 10 | 10 | 12 | 12 | 9 | 9 | 9 | 9 | 11 | 11 | 8 | 8 | 10 | 10 | 8 | 8 | 9 | 9 | 10 | 10 | NA | 6 | NA | 6 | NA | 6 | NA | 6 |
| Shigella sp. | 6 | 6 | 6 | 6 | 6 | 6 | 6 | 6 | 6 | 6 | 6 | 6 | 6 | 6 | 6 | 6 | 6 | 6 | 6 | 6 | NA | 6 | NA | 6 | NA | 6 | NA | 6 |
| Pseudomonas | 6 | 6 | 6 | 6 | 6 | 6 | 6 | 6 | 6 | 6 | 6 | 6 | 6 | 6 | 6 | 6 | 6 | 6 | 6 | 6 | NA | 6 | NA | 6 | NA | 6 | NA | 6 |
| sp. | | | | | | | | | | | | | | | | | | | | | | | | | | | | |
| Klebsiella sp. | 6 | 6 | 6 | 6 | 6 | 6 | 6 | 6 | 6 | 6 | 6 | 6 | 6 | 6 | 6 | 6 | 6 | 6 | 6 | 6 | NA | 6 | NA | 6 | NA | 6 | NA | 6 |
| Aeromonas sp. | 6 | 6 | 9 | 9 | 6 | 6 | 6 | 6 | 6 | 6 | 6 | 6 | 6 | 6 | 6 | 6 | 6 | 6 | 6 | 6 | NA | 6 | NA | 6 | NA | 6 | NA | 6 |
| Pleisiomonas | 10 | 10 | 11 | 11 | 9 | 9 | 6 | 6 | 6 | 6 | 6 | 6 | 10 | 10 | 6 | 6 | 6 | 6 | 6 | 6 | NA | 6 | NA | 10 | NA | 9 | NA | 6 |
| sp. | | | | | | | | | | | | | | | | | | | | | | | | | | | | |
| Candida | 6 | 6 | 6 | 6 | 6 | 6 | 6 | 6 | 6 | 6 | 6 | 6 | 6 | 6 | 6 | 6 | 6 | 6 | 6 | 6 | NA | 6 | NA | 6 | NA | 6 | NA | 6 |
| abicans | | | | | | | | | | | | | | | | | | | | | | | | | | | | |
| Vibrio cholera | 6 | 10 | 6 | 6 | 6 | 6 | 6 | 6 | 6 | 6 | 6 | 6 | 6 | 6 | 6 | 6 | 6 | 6 | 6 | 6 | NA | 6 | NA | 6 | NA | 6 | NA | 6 |
| Citrobacter sp. | 6 | 6 | 6 | 6 | 6 | 6 | 6 | 6 | 6 | 6 | 6 | 6 | 6 | 6 | 6 | 6 | 6 | 6 | 6 | 6 | NA | 6 | NA | 6 | NA | 6 | NA | 6 |

Values higher than 6 mm is indicative of antimicrobial activity. Staphylococcus, Proteus, Enteroaggregative E. coli (EAEC, Enterotoxigenic E. coli (ETEC), Aeromonas species, Plesiomonas species and Vibrio cholera showed positive inhibition.

We claim:

1. A method of inhibiting the growth of E. coli strains, said method comprising steps of bringing into contact E. coli strains with effective concentration of bioactive compounds isolated from Curcuma species and their derivatives, individually or in combination to bring about inhibition in the growth of E. coli

2. The method as in claim 1, wherein strains of E. coli include Enteroaggregative E. coli (EAEC) and Enterotoxigenic E. coli (ETEC).

3. The method as in claim 1, wherein the bioactive compounds and their derivatives for inhibiting the growth of EAEC are selected from the group consisting of Calebin A, O,O′-Diacetylcalebin-A, Diethylcalebin-A dicarbonate, and Tetrahydrocurcumin isoxazole.

4. The method as in claim 1, wherein the bioactive compounds and their derivatives for the inhibiting the growth of ETEC are selected from the group consisting of Curcumin, Demethoxycurcumin, Bis-demethoxycurcumin, O,O′-Diacetylcurcumin, Calebin A, O,O′-Diacetylcalebin-A, Diethylcalebin-A dicarbonate, Tetrahydrocurcumin isoxazole, Amino-tetrahydrocurcumin and Dibromocurcumin.

5. A method for the therapeutic management of infections caused by strains of E. coli in mammals, said method comprising step of administering an effective dose of bioactive compounds isolated from Curcuma species and their derivatives, individually or in combination, to said mammals to bring about reduction in the infection caused by E. coli strains.

6. The method as in claim 5, wherein the strains of E. coli include Enteroaggregative E. coli (EAEC) and Enterotoxigenic E. coli (ETEC).

7. The method as in claim 5, wherein the bioactive compounds and their derivatives for treating EAEC infections are selected from the group consisting of Calebin A, O,O′-Diacetylcalebin-A, Diethylcalebin-A dicarbonate, and Tetrahydrocurcumin isoxazole.

8. The method as in claim 5, wherein the bioactive compounds and their derivatives for treating ETEC infections are selected from the group consisting of Curcumin, Demethoxycurcumin, Bis-demethoxycurcumin, O,O′-Diacetylcurcumin, Calebin A, O,O′-Diacetylcalebin-A, Diethylcalebin-A dicarbonate, Tetrahydrocurcumin isoxazole, Amino tetrahydrocurcumin and Dibromocurcumin.

9. The method as in claim 5, wherein the infections caused by EAEC are selected from the group consisting of watery diarrhoea, mucoid diarrhoea, low-grade fever, nausea, tenesmus, and borborygmi.

10. The method as in claim 5, wherein the infections caused by ETEC are selected from the group consisting of severe diarrhoea, dysentery, abdominal cramps, and fever.

11. The method as in claim 5, wherein the mammal is human.

12. A method of inhibiting the growth of Pleisiomonas species, said method comprising steps of bringing into contact Pleisiomonas species with effective concentration of bioactive compounds isolated from Curcuma species and their derivatives, individually or in combination to bring about inhibition in the growth of Pleisiomonas species.

13. The method as in claim 12, wherein the bioactive compounds and their derivatives are selected from the group consisting of Curcumin, Demethoxycurcumin, Bis-demethoxycurcumin, Diethylcalebin-A dicarbonate, Demethoxycalebin-A, and Bisdemethoxycalebin-A.

14. A method for the therapeutic management of infections caused by strains of Pleisiomonas species in mammals, said method comprising step of administering an effective dose of bioactive compounds isolated from Curcuma species and their derivatives, individually or in combination, to said mammals to bring about reduction in the infection caused by Pleisiomonas species.

15. The method as in claim 14, wherein the bioactive compounds and their derivatives are selected from the group consisting of Curcumin, Demethoxycurcumin, Bis-demethoxycurcumin, Diethylcalebin-A dicarbonate, Demethoxycalebin-A, and Bisdemethoxycalebin-A.

16. The method as in claim 14, wherein the infections caused by Pleisiomonas species are selected from the group consisting of gastrointestinal infections, extraintestinal infections, gastroenteritis, watery diarrhea, abdominal pain, nausea and/or vomiting, headache, dehydration and fever.

17. The method as in claim 14, wherein the mammal is human.
